(19) United States
(12) Patent Application Publication
(10) Pub. No.: US 2008/0293722 A1
(43) Pub. Date: Nov. 27, 2008

(54) Ion Channel Modulators

(75) Inventors: Robert Zelle, Stow, MA (US); Christopher Todd Baker, Bedford, MA (US); Paul Will, Sudbury, MA (US); Vincent P. Galullo, South Grafton, MA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 10/592,893

(22) PCT Filed: Mar. 15, 2005
(86) PCT No.: PCT/US05/08761

§ 371 Date: Sep. 15, 2006

Related U.S. Application Data
(60) Provisional application No. 60/553,789, filed on Mar. 16, 2004.
(60) Provisional application No. 60/553,786, filed on Mar. 16, 2004.

(51) Int. Cl. A61K 31/155; A61P 9/00; A61P 11/00; A61P 25/00; C07C 257/14; C07D 295/14; C07D 307/22; A61K 31/495; C07D 211/26; C07D 213/58; A61K 31/5375; A61K 31/4453; A61K 31/341; A61K 31/44

(52) U.S. Cl. 514/238.5; 514/252.12; 564/246; 544/165; 544/336; 544/402; 546/231; 546/332; 549/491; 514/331; 514/471; 514/357; 514/637

(57) ABSTRACT

The invention relates to compounds, compositions comprising the compounds, and methods of using the compounds and compound compositions. The compounds, compositions, and methods described herein can be used for the therapeutic modulation of ion channel function, and treatment of disease and disease symptoms, particularly those mediated by certain calcium channel subtype targets.

### BACKGROUND

All cells rely on the regulated movement of inorganic ions across cell membranes to perform essential physiological functions. Electrical excitability, synaptic plasticity, and signal transduction are examples of processes in which changes in ion concentration play a critical role. In general, the ion channels that permit these changes are proteinaceous pores consisting of one or multiple subunits, each containing two or more membrane-spanning domains. Most ion channels have selectivity for specific ions, primarily Na⁺, K⁺, Ca²⁺, or Cl⁻, by virtue of physical preferences for size and charge. Electrochemical forces, rather than active transport, drive ions across membranes, thus a single channel may allow the passage of millions of ions per second. Channel opening, or “gating” is tightly controlled by changes in voltage or by ligand binding, depending on the subclass of channel. Ion channels are attractive therapeutic targets due to their involvement in so many physiological processes, yet the generation of drugs with specificity for particular channels in particular tissue types remains a major challenge.

Voltage-gated ion channels open in response to changes in membrane potential. For example, depolarization of excitable cells such as neurons result in a transient influx of Na⁺ ions, which propagates nerve impulses. This change in Na⁺ concentration is sensed by voltage-gated K⁺ channels, which then allow an efflux of K⁺ ions. The efflux of K⁺ ions repolarizes the membrane. Other cell types rely on voltage-gated Ca²⁺ channels to generate action potentials. Voltage-gated ion channels also perform important functions in non-excitable cells, such as the regulation of secretory, homeostatic, and mitogenic processes. Ligand-gated ion channels can be opened by extracellular stimuli such as neurotransmitters (e.g., glutamate, serotonin, acetylcholine), or intracellular stimuli (e.g. cAMP, Ca²⁺, and phosphorylation).

The Caᵥ1 family of voltage-gated calcium channels consists of 4 main subtypes Caᵥ1.1, Caᵥ1.2, Caᵥ1.3 and Caᵥ1.4. These currents are primarily found in skeletal muscle for Caᵥ1.1, heart, smooth muscle, brain, pituitary and adrenal tissue for Caᵥ1.2, brain pancreas, heart, kidney, ovary and cochlea for Caᵥ1.3 and in retina for Caᵥ1.4. These currents require a strong depolarization for activation and are long lasting. The subunit composition of the Caᵥ1 channels is defined by their α₁ subunit, which forms the pore and contains the voltage-sensing gates (α₁1.1, α₁1.2, α₁1.3 and α₁1.4, also known as α_{1S}, α_{1C}, α_{1D}, and α_{1F} respectively) and the β, α₂δ and γ subunits.

Genetic or pharmacological perturbations in ion channel function can have dramatic clinical consequences. Long QT syndrome, epilepsy, cystic fibrosis, and episodic ataxia are a few examples of heritable diseases resulting from mutations in ion channel subunits. Toxic side affects such as arrhythmia and seizure which are triggered by certain drugs are due to interference with ion channel function (Sirois, J. E. and, Atchison, W. D., Neurotoxicology 1996; 17(1):63-84; Keating, M. T., Science 1996272:681-685). Drugs are useful for the therapeutic modulation of ion channel activity, and have applications in treatment of many pathological conditions, including hypertension, angina pectoris, myocardial ischemia, asthma, bladder overactivity, alopecia, pain, heart failure, dysmenorrhea, type II diabetes, arrhythmia, graft rejection, seizure, convulsions, epilepsy, stroke, gastric hypermotility, psychoses, cancer, muscular dystrophy, and narcolepsy (Coghlan, M. J., et al. J. Med. Chem. 2001, 44:1627-1653; Ackerman. M. J., and Clapham, D. E. N. Eng. J. Med. 1997, 336:1575-1586). The growing number of identified ion channels and understanding of their complexity will assist in future efforts at therapies, which modify ion channel function.

Overactive bladder (OAB) is characterized by storage symptoms such as urgency, frequency and nocturia, with or without urge incontinence, resulting from the overactivity of the detrusor muscle in the bladder. OAB can lead to urge incontinence. The etiology of OAB and painful bladder syndrome is unknown, although disturbances in nerves, smooth muscle and urothelium can cause OAB (Steers, W. Rev Urol, 4:S7-S18). There is evidence to suggest that reduction of bladder hyperactivity may be indirectly effected by inhibition of Caᵥ2.2 and/or Caᵥ1 channels.

### SUMMARY

The invention relates to heterocyclic compounds, compositions comprising the compounds, and methods of using the compounds and compound compositions. The compounds and compositions comprising them are useful for treating disease or disease symptoms, including those mediated by or associated with ion channels.

In one aspect is a compound of formula (I) or pharmaceutical salt thereof

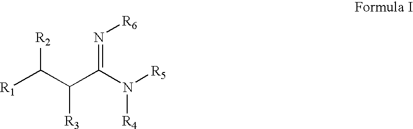

wherein,
R¹ is (CH₂)ₘAr¹;
each Ar¹ is independently aryl, heteroaryl, heterocyclyl or cycloalkyl, each optionally substituted with one or more R¹⁰;
each m is 0, 1, 2, 3, 4 or 5;
each R³ is independently (CH₂)ₚAr²;
p is 0, 1 or 2;
each Ar² is independently aryl, or heteroaryl, each optionally substituted with one or more R¹⁰;
R² is independently H;
each R⁴ is independently H, alkyl, (CH₂)ₘZ, or C(O)R⁵;
each Z is independently OCH₂CH₂OH, NR⁷R⁸, OR⁵, or Ar³;
each Ar³ is independently cycloalkyl, heterocyclyl, aryl, or heteroaryl, each optionally substituted with one or more R¹⁰;
or R⁴ and R⁵ taken together with the nitrogen atom to which they are attached form a 3 to 6 membered-ring, having carbon atoms and optionally in addition to the aforementioned nitrogen atom 1 or 2 additional heteroatoms that are NR¹¹, O or S, wherein the ring formed by R⁴ and R⁵ can be substituted by 1-3 R¹⁰;
each R⁵ is independently H or lower alkyl;
each R⁶ is independently H or lower alkyl;
or R⁵ and R⁶ taken together are —(CR¹²R¹³)ₙ—, where n is 2 or 3;
each R⁷ is independently hydrogen or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁸ is independently hydrogen, (CH₂)_{q}Ar⁴, or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁹ is independently (CH₂)_{q}Ar⁴ or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each Ar⁴ is independently aryl or heteroaryl, each optionally substituted with one to three substituents independently selected from halogen, OH, C₁-C₄ alkoxy, NH₂, C₁-C₄ alkylamino, C₁-C₄ dialkylamino or C₃-C₆ cycloalkyl;
each q is 0 or 1; and
each R¹⁰ is independently halogen, CN, NO₂, OR⁷, SR⁷, S(O)₂OR⁷, NR⁷R⁸, alkyl, hydroxyalkyl, cycloalkyl, Ar⁴, Ar⁴alkyl, C₁-C₂ perfluoroalkyl, C₁-C₂ perfluoroalkoxy, oxo, 1,2-methylenedioxy, C(O)OR⁷, C(O)NR⁷R⁸, OC(O)NR⁷R⁸, NR⁷C(O)NR⁷R⁸, C(NR⁷)NR⁷R⁸, NR⁷C(NR⁸)NR⁷R⁸, S(O)₂NR⁷R⁸, R⁹, C(O)R⁹, NR⁷C(O)R⁹, S(O)R⁹, or S(O)₂R⁹;
each R¹¹ is independently alkyl, aryl or aralkyl, each optionally substituted with one or more R¹⁰;
each R¹² is independently H, alkyl, or aryl; and
each R¹³ is independently H, alkyl, or aryl.

In other aspects, the compounds are those of any of the formulae herein (including any combinations thereof):

Wherein,
R¹ is (CH₂)aryl, optionally substituted by one or more R¹⁰;
R³ is aryl, optionally substituted by one or more R¹⁰;
R⁴ is (CH₂)ₘZ;
R⁵ is H; and
R⁶ is H;

Wherein,
Z is independently Ar³;

Wherein,
Ar³ is independently heterocyclyl optionally substituted with one or more R¹⁰ (e.g., 1, 2, 3, or 4);

Wherein,
Ar³ is independently heteroaryl optionally substituted with one or more R¹⁰;

Wherein,
Ar³ is independently aryl optionally substituted with one or more R¹⁰;

Wherein,
R¹ is (CH₂)Ar¹;
R³ is aryl or heteroaryl, each optionally substituted with one or more R¹⁰;
R⁴ is (CH₂)ₘZ;
R⁵ is H; and
R⁶ is H;

Wherein,
R¹ is Ar¹;
R³ is aryl or heteroaryl, each optionally substituted with one or more R¹⁰;
R⁴ is (CH₂)ₘZ;
R⁵ is H; and
R⁶ is H;

Wherein,
Ar¹ is heteroaryl optionally substituted with one or more R¹⁰;

Wherein,
Ar¹ is aryl optionally substituted with one or more R¹⁰;

Wherein,
R⁴ and R⁵ taken together with the nitrogen atom to which they are attached form a 3 to 6 membered-ring, having carbon atoms and optionally in addition to the aforementioned nitrogen atom 1 or 2 additional heteroatoms that are NR¹¹, O or S, wherein the ring formed by R⁴ and R⁵ can be substituted by 1-3 R¹⁰;

Wherein,
R⁵ and R⁶ taken together are —(CR¹²R¹³)ₙ—, where n is 2 or 3;

Wherein,
R¹ is Ar¹ or (CH₂)Ar¹; and
R³ is aryl or heteroaryl, each optionally substituted with one or more R¹⁰.

In another aspect is a compound of formula (I) or pharmaceutical salt thereof

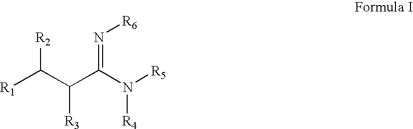

wherein,
R¹ is (CH₂)ₘAr¹;
each Ar¹ is independently aryl, heteroaryl, heterocyclyl or cycloalkyl, each optionally substituted with one or more R¹⁰;
each m is 0, 1, 2, 3, 4 or 5;
each R² is independently (CH₂)ₚAr²;
p is 0, 1 or 2;
each Ar² is independently aryl, or heteroaryl, each optionally substituted with one or more R¹⁰;
R³ is independently H;
each R⁴ is independently H, alkyl, (CH₂)ₘZ, or C(O)R⁵;
each Z is independently OCH₂CH₂OH, NR⁷R⁸, OR⁵, or Ar³;
each Ar³ is independently cycloalkyl, heterocyclyl, aryl, or heteroaryl, each optionally substituted with one or more R¹⁰;
or R⁴ and R⁵ taken together with the nitrogen atom to which they are attached form a 3 to 6 membered-ring, having carbon atoms and optionally in addition to the aforementioned nitrogen atom 1 or 2 additional heteroatoms that are NR¹¹, O or S, wherein the ring formed by R⁴ and R⁵ can be substituted by 1-3 R¹⁰;
each R⁵ is independently H or lower alkyl;
each R⁶ is independently H or lower alkyl;
or R⁵ and R⁶ taken together are —(CR¹²R¹³)ₙ—, where n is 2 or 3;
each R⁷ is independently hydrogen or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁸ is independently hydrogen, (CH₂)_{q}Ar⁴, or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁹ is independently (CH₂)_{q}Ar⁴ or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each Ar⁴ is independently aryl or heteroaryl, each optionally substituted with one to three substituents independently selected from halogen, OH, C₁-C₄ alkoxy, NH₂, C₁-C₄ alkylamino, C₁-C₄ dialkylamino or C₃-C₆ cycloalkyl;
each q is 0 or 1; and
each R¹⁰ is independently halogen, CN, NO₂, OR⁷, SR⁷, S(O)₂OR⁷, NR⁷R⁸, alkyl, hydroxyalkyl, cycloalkyl, Ar⁴, Ar⁴alkyl, C₁-C₂ perfluoroalkyl, C₁-C₂ perfluoroalkoxy, oxo, 1,2-methylenedioxy, C(O)OR⁷, C(O)NR⁷R⁸, OC(O)NR⁷R⁸, NR⁷C(O)NR⁷R⁸, C(NR⁷)NR⁷R⁸, NR⁷C(NR⁸)NR⁷R⁸, S(O)₂NR⁷R⁸, R⁹, C(O)R⁹, NR⁷C(O)R⁹, S(O)R⁹, or S(O)₂R⁹;
each R¹¹ is independently alkyl, aryl or aralkyl, each optionally substituted with one or more R¹⁰;
each R¹² is independently H, alkyl, or aryl; and
each R¹³ is independently H, alkyl, or aryl.

In yet other aspects, the compounds are those of any of the formulae herein (including any combinations thereof):

Wherein,
R¹ is aryl, optionally substituted by one or more R¹⁰;
R² is aryl, optionally substituted by one or more R¹⁰;
R⁴ is (CH₂)ₘZ;
R⁵ is H; and
R⁶ is H;

Wherein,
Z is independently Ar³;

Wherein,
Ar³ is independently heterocyclyl optionally substituted with one or more R¹⁰;

Wherein,
Ar³ is independently heteroaryl optionally substituted with one or more R¹⁰;

Wherein,
Ar³ is independently aryl optionally substituted with one or more R¹⁰;

Wherein,
R¹ is (CH₂)Ar¹;
R² is aryl or heteroaryl, each optionally substituted with one or more R¹⁰;
R⁴ is (CH₂)ₘZ;
R⁵ is H; and
R⁶ is H;

Wherein,
R¹ is Ar¹;
R² is aryl or heteroaryl, each optionally substituted with one or more R¹⁰;
R⁴ is (CH₂)ₘZ;
R⁵ is H; and
R⁶ is H;

Wherein,
Ar¹ is heteroaryl optionally substituted with one or more R¹⁰;

Wherein,
Ar¹ is aryl optionally substituted with one or more R¹⁰;

Wherein,
R⁴ and R⁵ taken together with the nitrogen atom to which they are attached form a 3 to 6 membered-ring, having carbon atoms and optionally in addition to the aforementioned nitrogen atom 1 or 2 additional heteroatoms that are NR¹¹, O or S, wherein the ring formed by R⁴ and R⁵ can be substituted by 1-3 R¹⁰

Wherein,
R⁵ and R⁶ taken together are —(CR¹²R¹³)ₙ—, where n is 2 or 3;

Wherein,
R¹ is Ar¹ or (CH₂)Ar¹; and
R² is aryl or heteroaryl, each optionally substituted with one or more R¹⁰;
   Wherein, the compound of formula I is a compound delineated in any of the tables herein or pharmaceutical salt thereof.

In other aspects, the invention relates to a composition comprising a compound of any of the formulae herein, an additional therapeutic agent, and a pharmaceutically acceptable carrier. The additional therapeutic agent can be a cardiovascular disease agent and/or a nervous system disease agent. A nervous system disease agent refers to a peripheral nervous system (PNS) disease agent and/or a central nervous system (CNS) disease agent.

Yet another aspect of this invention relates to a method of treating a subject (e.g., mammal, human, horse, dog, cat) having a disease or disease symptom (including, but not limited to angina, hypertension, congestive heart failure, myocardial ischemia, atrial fibrillation, diabetes mellitus, urinary incontinence, overactive bladder, pulmonary disease, cognitive function, or a nervous system disorder). The method includes administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

Yet another aspect of this invention relates to a method of treating a subject (e.g., mammal, human, horse, dog, cat) having an ion channel mediated disease or disease symptom (including, but not limited to angina, hypertension, congestive heart failure, myocardial ischemia, atrial fibrillation, diabetes mellitus, urinary incontinence, overactive bladder, pulmonary disease, cognitive function, or a nervous system disorder). The method includes administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

Another aspect is a method of modulating (e.g., inhibiting, agonism, antagonism) calcium channel activity comprising contacting a calcium channel with a compound (or composition thereof) of any of the formulae herein.

Other aspects are a method of modulating calcium channel Caᵥ1 (e.g., Cav1.2, Cav1.3) activity in a subject in need thereof including administering to the subject a therapeutically effective amount of a compound (or composition thereof) of any of the formulae herein.

The invention also relates to a method of making a compound described herein, the method including any reactions or reagents as delineated in the schemes or examples herein. Alternatively, the method includes taking any one of the intermediate compounds described herein and reacting it with one or chemical reagents in one or more steps to produce a compound described herein.

Also within the scope of this invention is a packaged product. The packaged product includes a container, one of the aforementioned compounds in the container, and a legend (e.g., a label or an insert) associated with the container and indicating administration of the compound for treating a disorder associated with ion channel modulation.

In other embodiments, the compounds, compositions, and methods delineated herein are any of the compounds of Table 1 herein or methods including them.

The details of one or more embodiments of the invention are set forth in the accompanying drawings and the description below. Other features, objects, and advantages of the invention will be apparent from the description and from the claims.

### DETAILED DESCRIPTION

As used herein, the term “halo” refers to any radical of fluorine, chlorine, bromine or iodine.

The term “alkyl” refers to a hydrocarbon chain that may be a straight chain or branched chain, containing the indicated number of carbon atoms. For example, C₁-C₅ indicates that the group may have from 1 to 5 (inclusive) carbon atoms in it. The term “lower alkyl” refers to a C₁-C₆ alkyl chain. The term “arylalkyl” refers to a moiety in which an alkyl hydrogen atom is replaced by an aryl group.

The term “alkoxy” refers to an —O-alkyl radical. The term “alkylene” refers to a divalent alkyl (i.e., —R—). The term “alkylenedioxo” refers to a divalent species of the structure —O—R—O—, in which R represents an alkylene.

The term “cycloalkyl” as employed herein includes saturated and partially unsaturated cyclic hydrocarbon groups having 3 to 12 carbons, preferably 3 to 8 carbons, and more preferably 3 to 6 carbon.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent.

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted by substituents.

The term “substituents” refers to a group “substituted” on an alkyl, cycloalkyl, aryl, heterocyclyl, or heteroaryl group at any atom of that group. Suitable substituents include, without limitation halogen, CN, NO₂, OR⁵, SR⁵, S(O)₂OR⁵, NR⁵R⁶, C₁-C₂ perfluoroalkyl, oxo, C₁-C₂ perfluoroalkoxy, 1,2-methylenedioxy, C(O)OR⁵, C(O)NR⁵R⁶, OC(O)NR⁵R⁶, NR⁵C(O)NR⁵R⁶, C(NR⁶)NR⁵R⁶, NR⁵C(NR⁶)NR⁵R⁶, S(O)₂NR⁵R⁶, R⁷, R⁷alkyl, C(O)R⁷, NR⁵C(O)R⁷, S(O)R⁷, or S(O)₂R⁷. Each R⁵ is independently hydrogen, C₁-C₄ alkyl or C₃-C₆ cycloalkyl. Each R⁶ is independently hydrogen, C₃-C₆ cycloalkyl, aryl, heterocyclyl, heteroaryl, C₁-C₄ alkyl or C₁-C₄ alkyl substituted with C₃-C₆ cycloalkyl, aryl, heterocyclyl or heteroaryl. Each R⁷ is independently C₃-C₆ cycloalkyl, aryl, heterocyclyl, heteroaryl, C₁-C₄ alkyl or C₁-C₄ alkyl substituted with C₃-C₆ cycloalkyl, aryl, heterocyclyl or heteroaryl. Each C₃-C₆ cycloalkyl, aryl, heterocyclyl, heteroaryl and C₁-C₄ alkyl in each R⁵, R⁶ and R⁷ can optionally be substituted with halogen, CN, C₁-C₄ alkyl, OH, C₁-C₄ alkoxy, NH₂, C₁-C₄ alkylamino, C₁-C₄ dialkylamino, C₁-C₂ perfluoroalkyl, C₁-C₂ perfluoroalkoxy, or 1,2-methylenedioxy.

In one aspect, the substituents on a group are independently, hydrogen, hydroxyl, halogen, nitro, SO₃H, trifluoromethyl, trifluoromethoxy, alkyl (C₁-C₆ straight or branched), alkoxy (C₁-C₆ straight or branched), O-benzyl, O-phenyl, phenyl, 1,2-methylenedioxy, carboxyl, morpholinyl, piperidinyl, amino or OC(O)NR⁵R⁶. Each R⁵ and R⁶ is as described above.

The term “treating” or “treated” refers to administering a compound described herein to a subject with the purpose to cure, heal, alleviate, relieve, alter, remedy, ameliorate, improve, or affect a disease, the symptoms of the disease or the predisposition toward the disease.

“An effective amount” refers to an amount of a compound, which confers a therapeutic effect on the treated subject. The therapeutic effect may be objective (i.e., measurable by some test or marker) or subjective (i.e., subject gives an indication of or feels an effect). An effective amount of the compound described above may range from about 0.1 mg/Kg to about 500 mg/Kg. Effective doses will also vary depending on route of administration, as well as the possibility of co-usage with other agents.

Representative compounds useful in the compositions and methods are delineated herein:

TABLE 1A

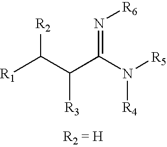

| No. | R₁ | R₃ | R₄ | R₅ | R₆ |
|---|---|---|---|---|---|
| 1 | 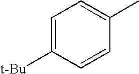 | 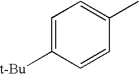 | H | —CH₂CH₂— | |
| 2 | 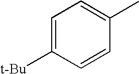 | 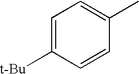 | H | H | H |
| 3 | 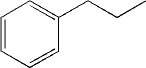 | 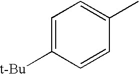 | H | H | H |
| 4 | 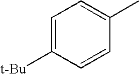 | 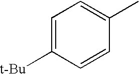 | Et | H | H |
| 5 | 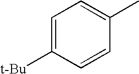 | 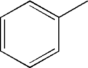 | H | H | H |
| 6 | 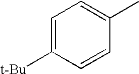 | 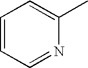 | H | H | H |
| 7 | 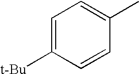 | 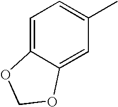 | H | H | H |
| 8 | 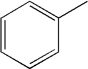 | 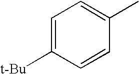 | H | —CH₂CH₂— | |
| 9 | 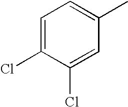 | 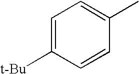 | H | —CH₂CH₂— | |
| 10 | 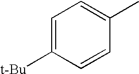 | 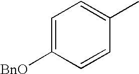 | H | H | H |
| 11 | 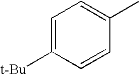 | 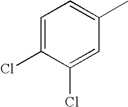 | H | H | H |
| 12 | 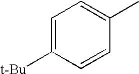 | 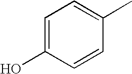 | H | H | H |
| 13 | 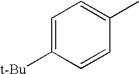 | 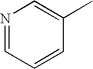 | H | —CH₂CH₂— | |
| 14 | 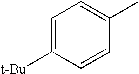 | 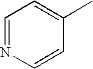 | H | H | H |
| 15 | 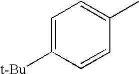 | 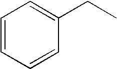 | H | H | H |
| 16 | 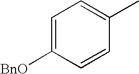 | 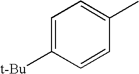 | H | H | H |
| 17 | 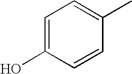 | 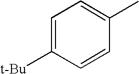 | H | H | H |
| 18 | 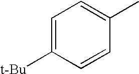 | 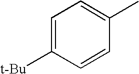 | 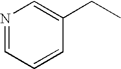 | H | H |
| 19 | 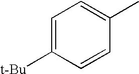 | 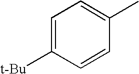 | 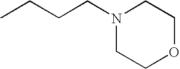 | H | H |
| 20 | 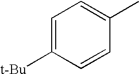 | 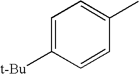 | 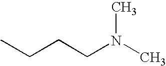 | H | H |
| 21 | 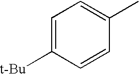 | 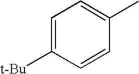 | —CH₂CH₂NHCH₂CH₂— | | H |
| 22 | 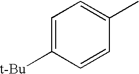 | 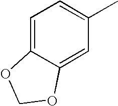 | 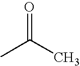 | H | H |
| 23 | 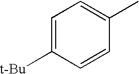 | 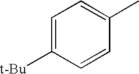 | H | —CH₂CH₂CH₂— | |
| 24 | 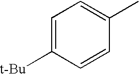 | 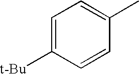 | 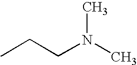 | H | H |
| 25 | 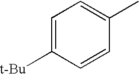 | 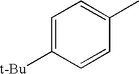 | H | 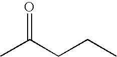 | |
| 26 | 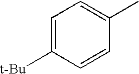 | 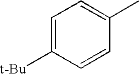 | 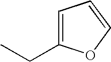 | H | H |
| 27 | 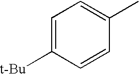 | 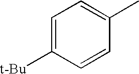 | 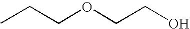 | H | H |
| 28 | 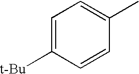 | 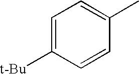 | 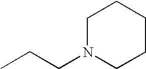 | H | H |
| 29 | 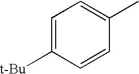 | 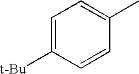 | 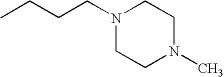 | H | H |
| 30 | 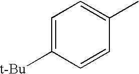 | 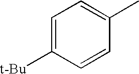 | 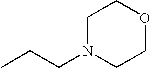 | H | H |
| 31 | 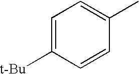 | 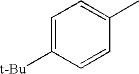 | 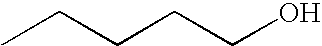 | H | H |
| 32 | 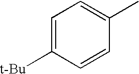 | 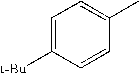 | 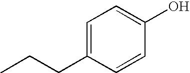 | H | H |
| 33 | 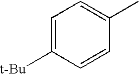 | 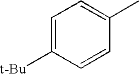 | 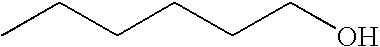 | H | H |
| 34 | 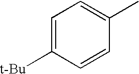 | 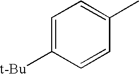 | 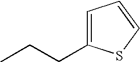 | H | H |
| 35 | 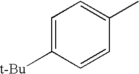 | 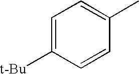 | 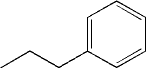 | H | H |
| 36 | 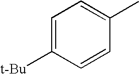 | 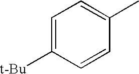 | 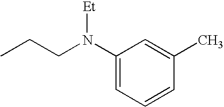 | H | H |
| 37 | 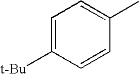 | 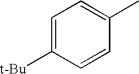 | 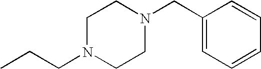 | H | H |
| 38 | 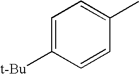 | 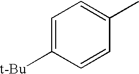 | 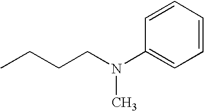 | H | H |
| 39 | 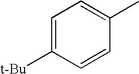 | 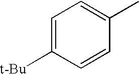 | 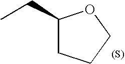 | H | H |
| 40 | 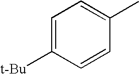 | 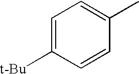 | 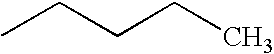 | H | H |
| 41 | 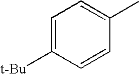 | 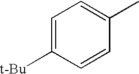 | 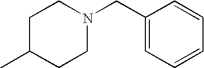 | H | H |
| 42 | 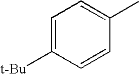 | 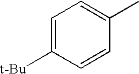 | 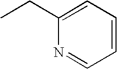 | H | H |
| 43 | 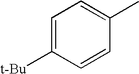 | 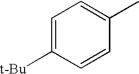 | 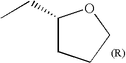 | H | H |
| 44 | 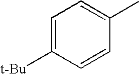 | 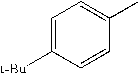 | 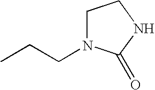 | H | H |
| 45 | 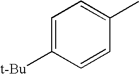 | 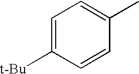 | 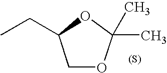 | H | H |
| 46 | 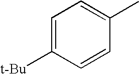 | 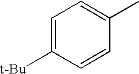 | 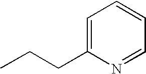 | H | H |
| 47 | 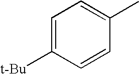 | 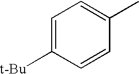 | 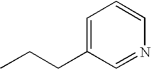 | H | H |
| 48 | 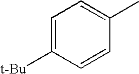 | 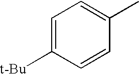 | 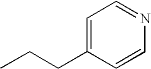 | H | H |
| 49 | 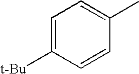 | 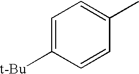 | 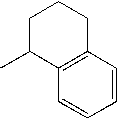 | H | H |
| 50 | 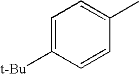 | 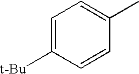 | 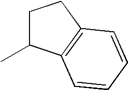 | H | H |
| 51 | 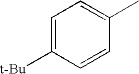 | 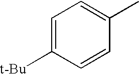 | 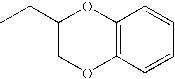 | H | H |
| 52 | 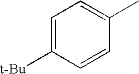 | 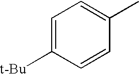 | 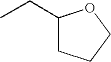 | H | H |
| 53 | 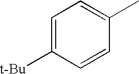 | 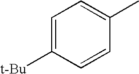 | i-Pr | H | H |
| 54 | 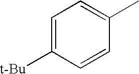 | 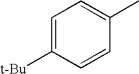 | 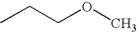 | H | H |
| 55 | 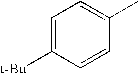 | 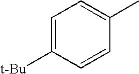 | 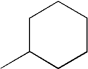 | H | H |
| 56 | 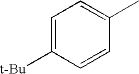 | 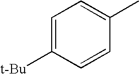 | t-Bu | H | H |
| 57 | 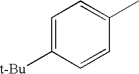 | 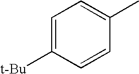 | 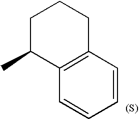 | H | H |
| 58 | 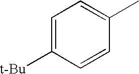 | 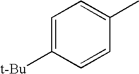 | 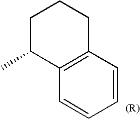 | H | H |
| 59 | 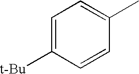 | 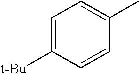 | 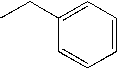 | H | H |
| 60 | 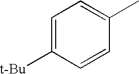 | 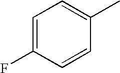 | 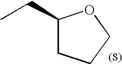 | H | H |
| 61 | 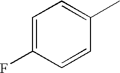 | 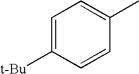 | 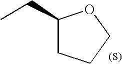 | H | H |
| 62 | 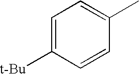 | 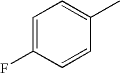 | CH₃ | H | H |
| 63 | 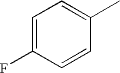 | 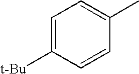 | CH₃ | H | H |
| 64 | 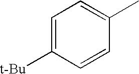 | 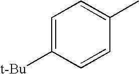 | CH₃ | H | H |
| 65 | 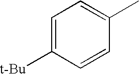 | 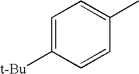 | 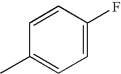 | H | H |
| 66 | 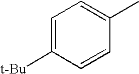 | 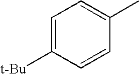 | 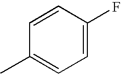 | H | H |
| 67 | 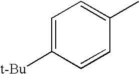 | 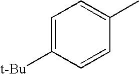 | H | 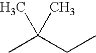 | |
| 68 | 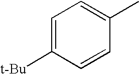 | 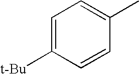 | H | 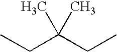 | |
| 69 | 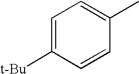 | 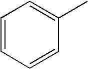 | H | —CH₂CH₂— | |
| 70 | 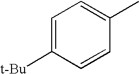 | 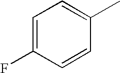 | H | —CH₂CH₂— | |
| 71 | 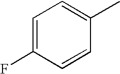 | 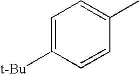 | H | —CH₂CH₂— | |
| 72 | 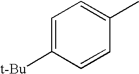 | 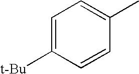 | CH₃ | CH₃ | CH₃ |
| 73 | 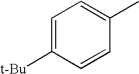 | 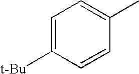 | CH₃ | CH₃ | H |
| 74 | 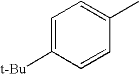 | 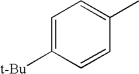 | —CH₂CH₂CH₂CH₂— | | H |
| 75 | 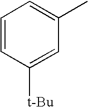 | 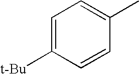 | H | H | H |
| 76 | 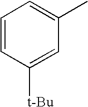 | 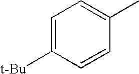 | 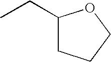 | H | H |
| 77 | 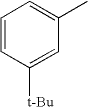 | 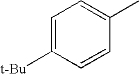 | 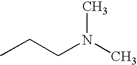 | H | H |
| 78 | 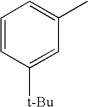 | 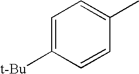 | 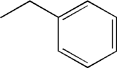 | H | H |
| 79 | 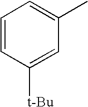 | 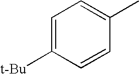 | H | —CH₂CH₂— | |
| 80 | 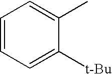 | 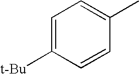 | H | H | H |
| 81 | 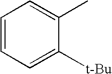 | 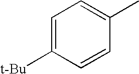 | 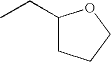 | H | H |
| 82 | 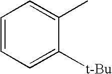 | 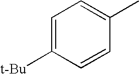 | 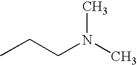 | H | H |
| 83 | 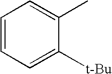 | 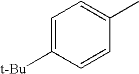 | 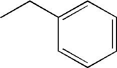 | H | H |
| 84 | 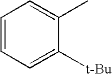 | 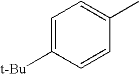 | H | —CH₂CH₂— | |
| 85 | 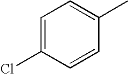 | 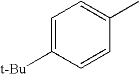 | H | H | H |
| 86 | 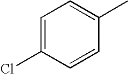 | 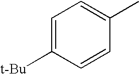 | 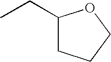 | H | H |
| 87 | 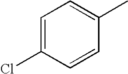 | 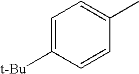 | 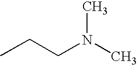 | H | H |
| 88 | 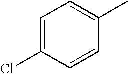 | 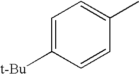 | 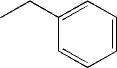 | H | H |
| 89 | 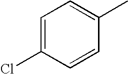 | 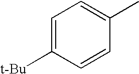 | H | —CH₂CH₂— | |
| 90 | 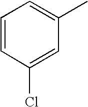 | 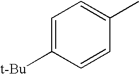 | H | H | H |
| 91 | 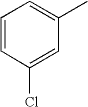 | 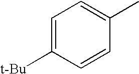 | 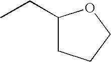 | H | H |
| 92 | 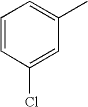 | 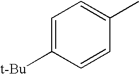 | 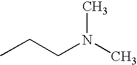 | H | H |
| 93 | 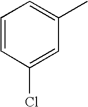 | 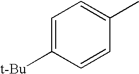 | 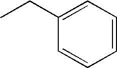 | H | H |
| 94 | 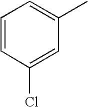 | 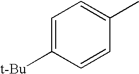 | H | —CH₂CH₂— | |
| 95 | 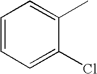 | 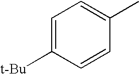 | H | H | H |
| 96 | 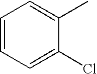 | 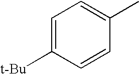 | 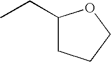 | H | H |
| 97 | 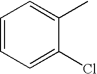 | 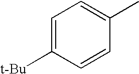 | 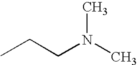 | H | H |
| 98 | 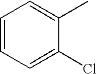 | 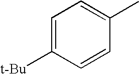 | 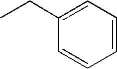 | H | H |
| 99 | 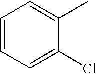 | 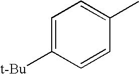 | H | —CH₂CH₂— | |
| 100 | 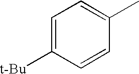 | 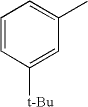 | H | H | H |
| 101 | 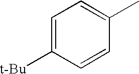 | 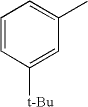 | 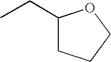 | H | H |
| 102 | 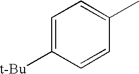 | 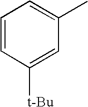 | 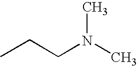 | H | H |
| 103 | 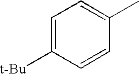 | 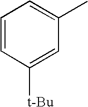 | 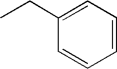 | H | H |
| 104 | 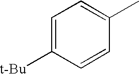 | 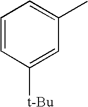 | H | —CH₂CH₂— | |
| 105 | 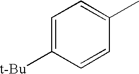 | 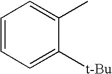 | H | H | H |
| 106 | 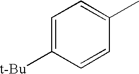 | 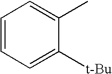 | 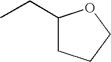 | H | H |
| 107 | 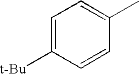 | 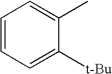 | 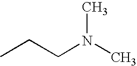 | H | H |
| 108 | 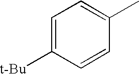 | 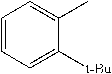 | 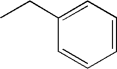 | H | H |
| 109 | 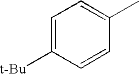 | 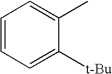 | H | —CH₂CH₂— | |
| 110 | 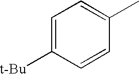 | 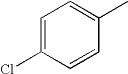 | H | H | H |
| 111 | 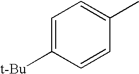 | 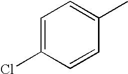 | 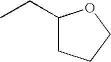 | H | H |
| 112 | 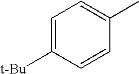 | 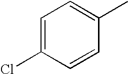 | 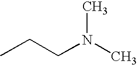 | H | H |
| 113 | 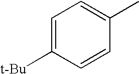 | 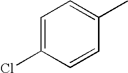 | 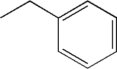 | H | H |
| 114 | 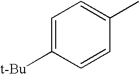 | 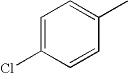 | H | —CH₂CH₂— | |
| 115 | 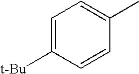 | 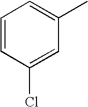 | H | H | H |
| 116 | 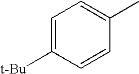 | 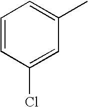 | 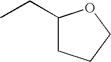 | H | H |
| 117 | 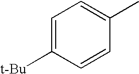 | 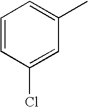 | 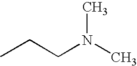 | H | H |
| 118 | 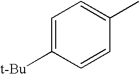 | 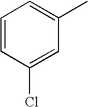 | 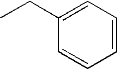 | H | H |
| 119 | 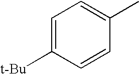 | 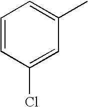 | H | —CH₂CH₂— | |
| 120 | 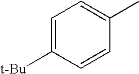 | 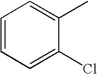 | H | H | H |
| 121 | 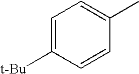 | 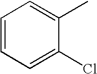 | 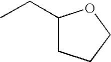 | H | H |
| 122 | 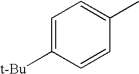 | 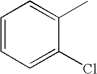 | 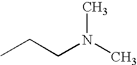 | H | H |
| 123 | 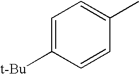 | 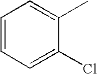 | 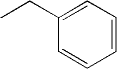 | H | H |
| 124 | 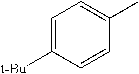 | 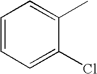 | H | —CH₂CH₂— | |
| 125 | 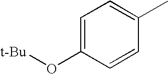 | 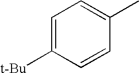 | H | H | H |
| 126 | 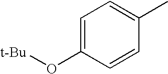 | 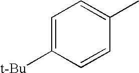 | 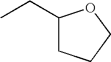 | H | H |
| 127 | 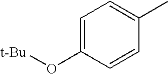 | 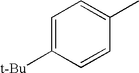 | 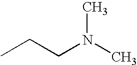 | H | H |
| 128 | 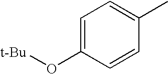 | 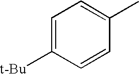 | 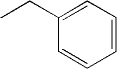 | H | H |
| 129 | 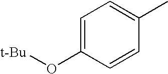 | 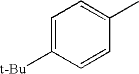 | H | —CH₂CH₂— | |
| 130 | 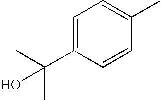 | 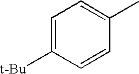 | H | H | H |
| 131 | 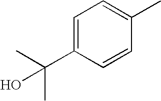 | 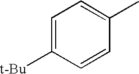 | 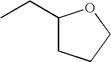 | H | H |
| 132 | 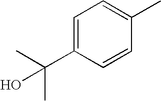 | 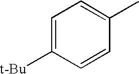 | 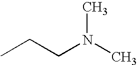 | H | H |
| 133 | 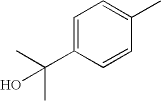 | 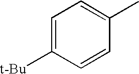 | 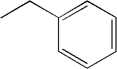 | H | H |
| 134 | 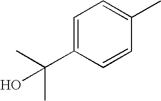 | 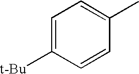 | H | —CH₂CH₂— | |
| 135 | 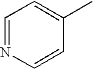 | 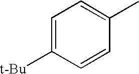 | H | H | H |
| 136 | 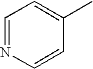 | 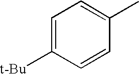 | 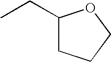 | H | H |
| 137 | 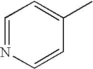 | 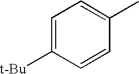 | 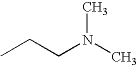 | H | H |
| 138 | 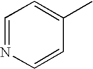 | 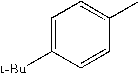 | 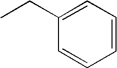 | H | H |
| 139 | 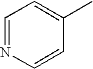 | 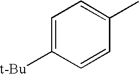 | H | —CH₂CH₂— | |
| 140 | 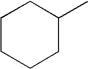 | 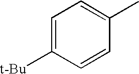 | H | H | H |
| 141 | 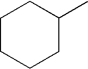 | 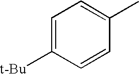 | 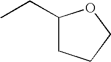 | H | H |
| 142 | 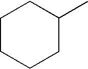 | 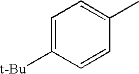 | 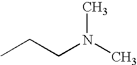 | H | H |
| 143 | 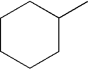 | 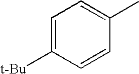 | 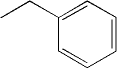 | H | H |
| 144 | 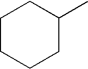 | 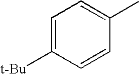 | H | —CH₂CH₂— | |
| 145 | 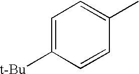 | 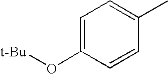 | H | H | H |
| 146 | 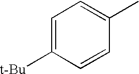 | 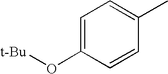 | 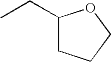 | H | H |
| 147 | 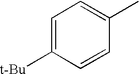 | 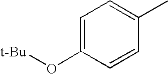 | 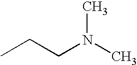 | H | H |
| 148 | 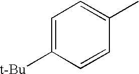 | 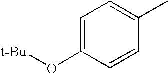 | 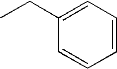 | H | H |
| 149 | 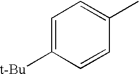 | 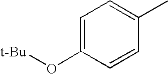 | H | —CH₂CH₂— | |
| 150 | 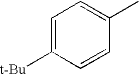 | 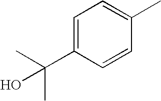 | H | H | H |
| 151 | 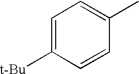 | 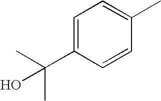 | 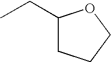 | H | H |
| 152 | 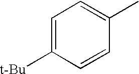 | 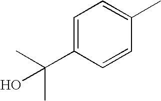 | 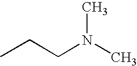 | H | H |
| 153 | 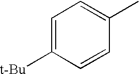 | 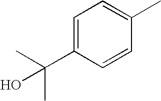 | 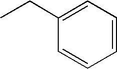 | H | H |
| 154 | 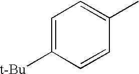 | 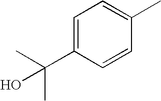 | H | —CH₂CH₂— | |
| 155 | 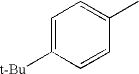 | 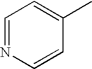 | 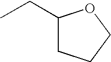 | H | H |
| 156 | 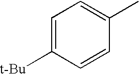 | 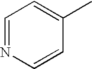 | 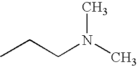 | H | H |
| 157 | 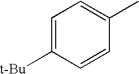 | 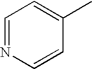 | 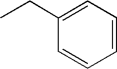 | H | H |
| 158 | 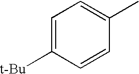 | 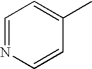 | H | —CH₂CH₂— | |
| 159 | 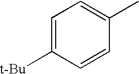 | 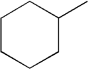 | H | H | H |
| 160 | 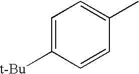 | 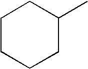 | 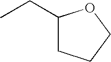 | H | H |
| 161 | 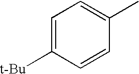 | 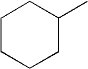 | 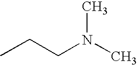 | H | H |
| 162 | 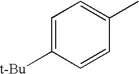 | 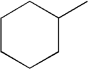 | 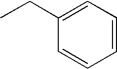 | H | H |

| | | | | |
|---|---|---|---|---|
| 163 | 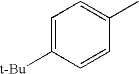 | 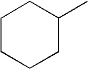 | H | —CH₂CH₂— |

TABLE 1B

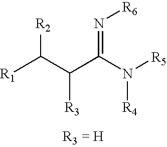

| No. | R₁ | R₂ | R₄ | R₅ | R₆ |
|---|---|---|---|---|---|
| 164 | 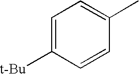 | 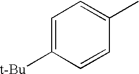 | H | H | H |
| 165 | 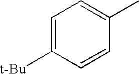 | 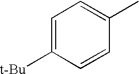 | 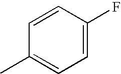 | H | H |
| 166 | 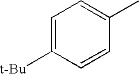 | 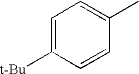 | 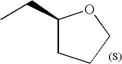 | H | H |
| 167 | 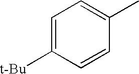 | 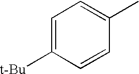 | H | —CH₂CH₂— | |
| 168 | 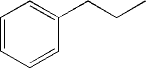 | 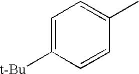 | H | H | H |
| 169 | 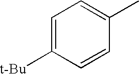 | 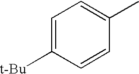 | Et | H | H |
| 170 | 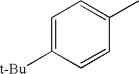 | 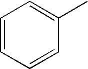 | H | H | H |
| 171 | 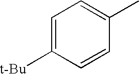 | 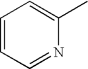 | H | H | H |
| 172 | 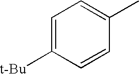 | 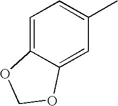 | H | H | H |
| 173 | 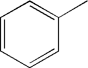 | 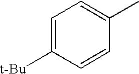 | H | —CH₂CH₂— | |
| 174 | 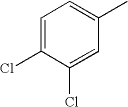 | 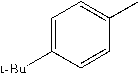 | H | —CH₂CH₂— | |
| 175 | 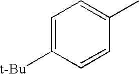 | 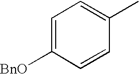 | H | H | H |
| 176 | 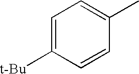 | 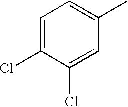 | H | H | H |
| 177 | 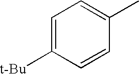 | 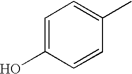 | H | H | H |
| 178 | 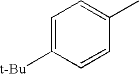 | 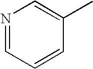 | H | —CH₂CH₂— | |
| 179 | 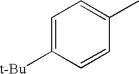 | 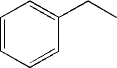 | H | H | H |
| 180 | 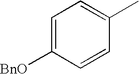 | 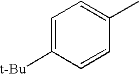 | H | H | H |
| 181 | 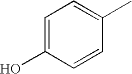 | 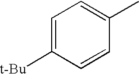 | H | H | H |
| 182 | 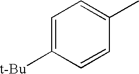 | 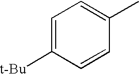 | 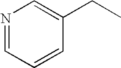 | H | H |
| 183 | 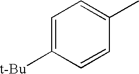 | 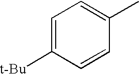 | 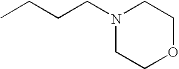 | H | H |
| 184 | 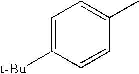 | 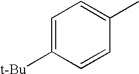 | 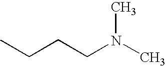 | H | H |
| 185 | 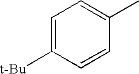 | 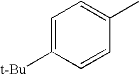 | —CH₂CH₂NHCH₂CH₂— | | H |
| 186 | 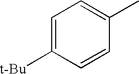 | 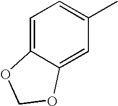 | 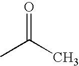 | H | H |
| 187 | 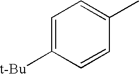 | 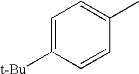 | H | —CH₂CH₂CH₂— | |
| 188 | 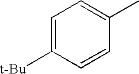 | 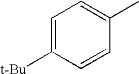 | 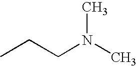 | H | H |
| 189 | 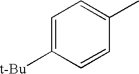 | 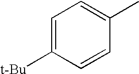 | H | 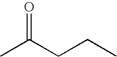 | |
| 190 | 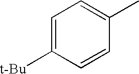 | 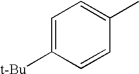 | 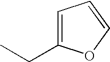 | H | H |
| 191 | 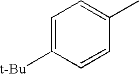 | 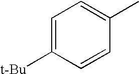 | 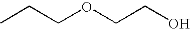 | H | H |
| 192 | 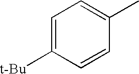 | 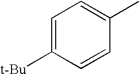 | 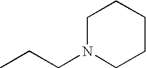 | H | H |
| 193 | 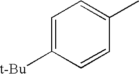 | 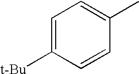 | 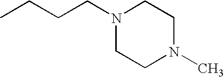 | H | H |
| 194 | 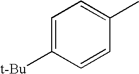 | 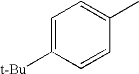 | 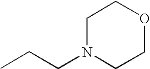 | H | H |
| 195 | 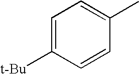 | 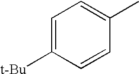 | 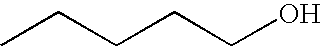 | H | H |
| 196 | 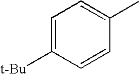 | 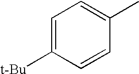 | 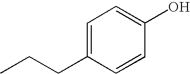 | H | H |
| 197 | 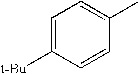 | 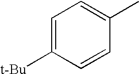 | 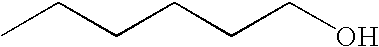 | H | H |
| 198 | 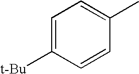 | 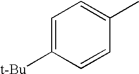 | 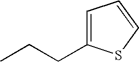 | H | H |
| 199 | 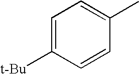 | 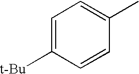 | 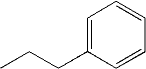 | H | H |
| 200 | 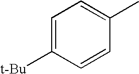 | 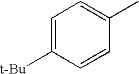 | 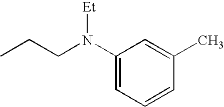 | H | H |
| 201 | 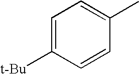 | 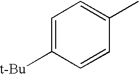 | 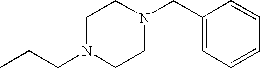 | H | H |
| 202 | 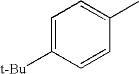 | 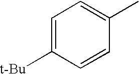 | 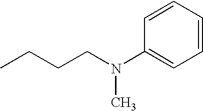 | H | H |
| 203 | 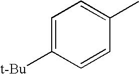 | 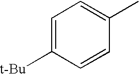 | 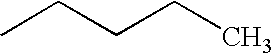 | H | H |
| 204 | 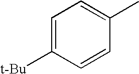 | 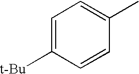 | 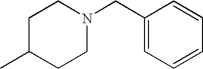 | H | H |
| 205 | 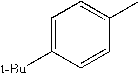 | 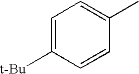 | 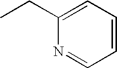 | H | H |
| 206 | 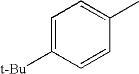 | 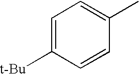 | 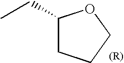 | H | H |
| 207 | 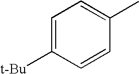 | 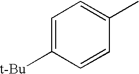 | 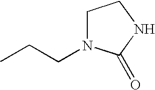 | H | H |
| 208 | 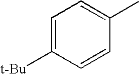 | 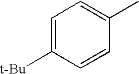 | 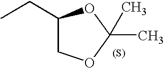 | H | H |
| 209 | 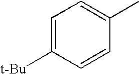 | 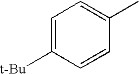 | 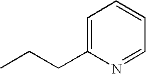 | H | H |
| 210 | 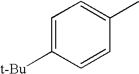 | 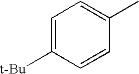 | 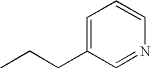 | H | H |
| 211 | 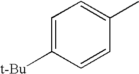 | 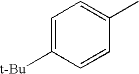 | 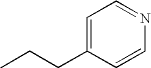 | H | H |
| 212 | 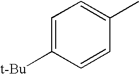 | 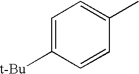 | 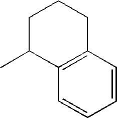 | H | H |
| 213 | 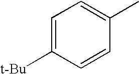 | 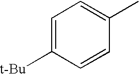 | 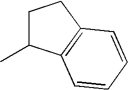 | H | H |
| 214 | 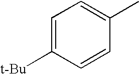 | 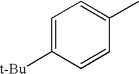 | 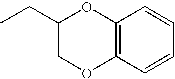 | H | H |
| 215 | 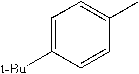 | 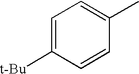 | 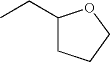 | H | H |
| 216 | 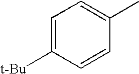 | 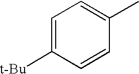 | i-Pr | H | H |
| 217 | 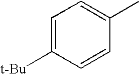 | 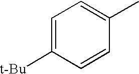 | 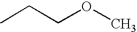 | H | H |
| 218 | 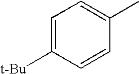 | 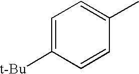 | 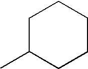 | H | H |
| 219 | 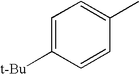 | 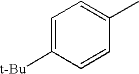 | t-Bu | H | H |
| 220 | 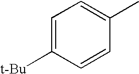 | 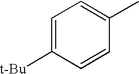 | 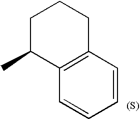 | H | H |
| 221 | 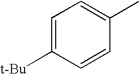 | 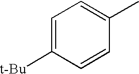 | 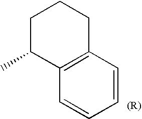 | H | H |
| 222 | 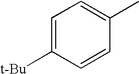 | 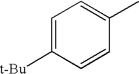 | 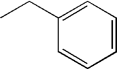 | H | H |
| 223 | 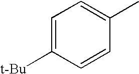 | 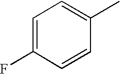 | 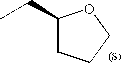 | H | H |
| 224 | 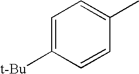 | 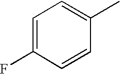 | CH₃ | H | H |
| 225 | 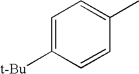 | 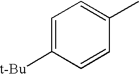 | CH₃ | H | H |
| 226 | 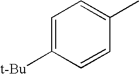 | 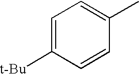 | 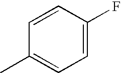 | CH₃ | H |
| 227 | 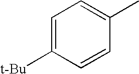 | 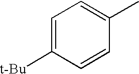 | H | 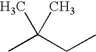 | |
| 228 | 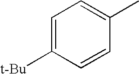 | 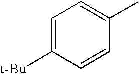 | H | 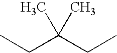 | |
| 229 | 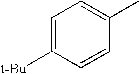 | 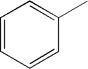 | H | —CH₂CH₂— | |
| 230 | 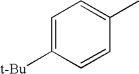 | 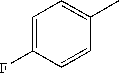 | H | —CH₂CH₂— | |
| 231 | 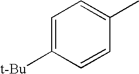 | 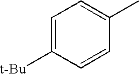 | CH₃ | CH₃ | CH₃ |
| 232 | 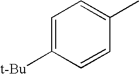 | 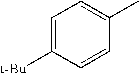 | CH₃ | CH₃ | H |
| 233 | 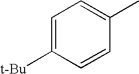 | 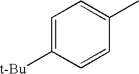 | —CH₂CH₂CH₂CH₂— | | H |
| 234 | 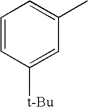 | 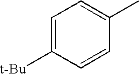 | H | H | H |
| 235 | 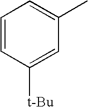 | 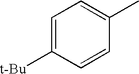 | 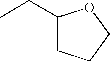 | H | H |
| 236 | 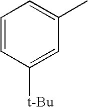 | 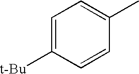 | 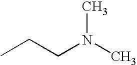 | H | H |
| 237 | 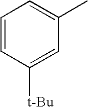 | 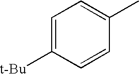 | 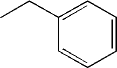 | H | H |
| 238 | 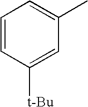 | 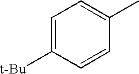 | H | —CH₂CH₂— | |
| 239 | 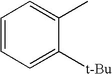 | 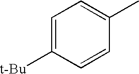 | H | H | H |
| 240 | 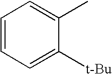 | 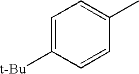 | 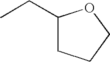 | H | H |
| 241 | 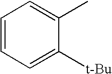 | 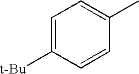 | 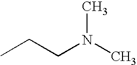 | H | H |
| 242 | 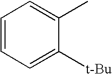 | 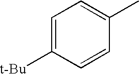 | 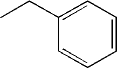 | H | H |
| 243 | 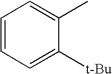 | 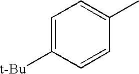 | H | —CH₂CH₂— | |
| 244 | 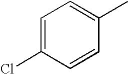 | 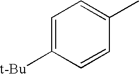 | H | H | H |
| 245 | 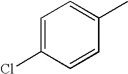 | 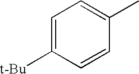 | 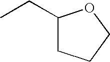 | H | H |
| 246 | 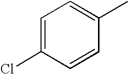 | 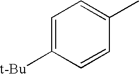 | 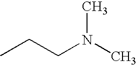 | H | H |
| 247 | 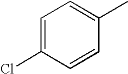 | 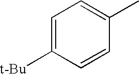 | 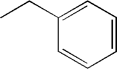 | H | H |
| 248 | 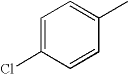 | 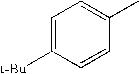 | H | —CH₂CH₂— | |
| 249 | 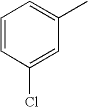 | 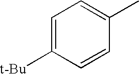 | H | H | H |
| 250 | 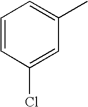 | 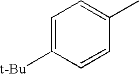 | 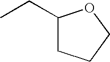 | H | H |
| 251 | 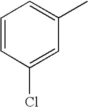 | 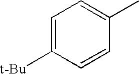 | 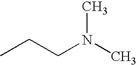 | H | H |
| 252 | 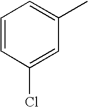 | 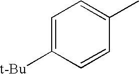 | 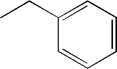 | H | H |
| 253 | 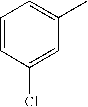 | 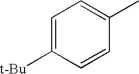 | H | —CH₂CH₂— | |
| 254 | 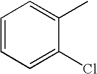 | 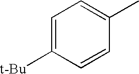 | H | H | H |
| 255 | 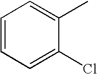 | 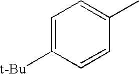 | 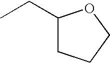 | H | H |
| 256 | 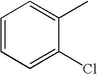 | 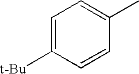 | 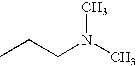 | H | H |
| 257 | 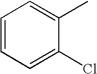 | 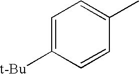 | 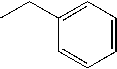 | H | H |
| 258 | 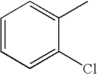 | 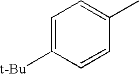 | H | —CH₂CH₂— | |
| 259 | 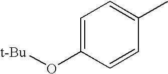 | 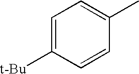 | H | H | H |
| 260 | 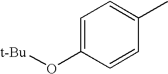 | 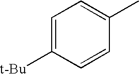 | 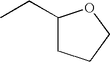 | H | H |
| 261 | 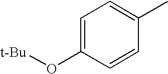 | 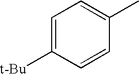 | 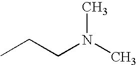 | H | H |
| 262 | 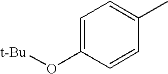 | 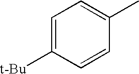 | 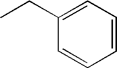 | H | H |
| 263 | 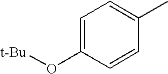 | 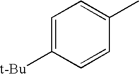 | H | —CH₂CH₂— | |
| 264 | 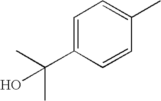 | 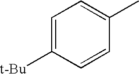 | H | H | H |
| 265 | 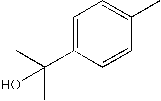 | 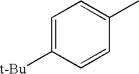 | 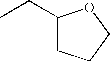 | H | H |
| 266 | 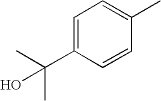 | 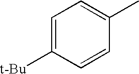 | 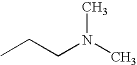 | H | H |
| 267 | 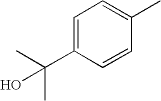 | 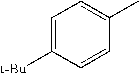 | 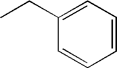 | H | H |
| 268 | 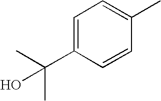 | 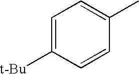 | H | —CH₂CH₂— | |
| 269 | 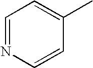 | 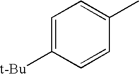 | H | H | H |
| 270 | 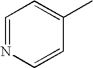 | 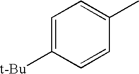 | 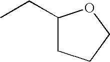 | H | H |
| 271 | 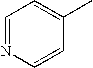 | 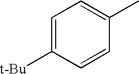 | 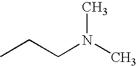 | H | H |
| 272 | 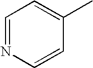 | 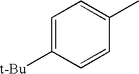 | 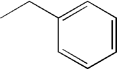 | H | H |
| 273 | 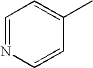 | 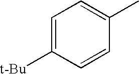 | H | —CH₂CH₂— | |
| 274 | 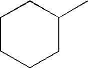 | 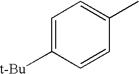 | H | H | H |
| 275 | 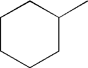 | 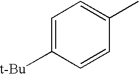 | 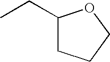 | H | H |
| 276 | 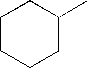 | 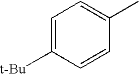 | 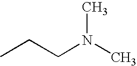 | H | H |
| 277 | 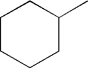 | 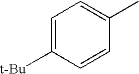 | 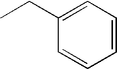 | H | H |

| | | | | |
|---|---|---|---|---|
| 278 | 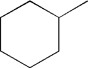 | 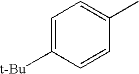 | H | —CH₂CH₂— |

Ion channel-modulating compounds can be identified through both in vitro (e.g., cell and non-cell based) and in vivo methods. Representative examples of these methods are described in the Examples herein.

Combinations of substituents and variables envisioned by this invention are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The compounds delineated herein can be synthesized using conventional methods, as illustrated in the schemes herein. Variables in the structures are defined as in any of the formulae herein, except where defined otherwise in the schemes.

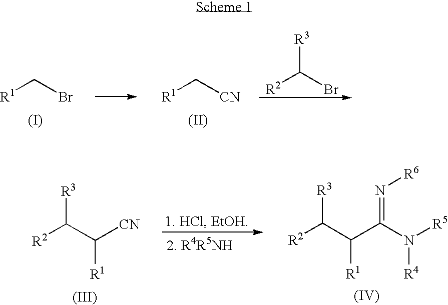

Treatment of a bromide (I) with potassium cyanide affords acetonitrile derivative (II). Formation of the anion of (II) under basic conditions and reaction with a bromide gives nitrile (III). Treatment of nitrile (III) with an alcohol under acidic conditions provides the alkoxy imidate intermediate, which is treated with the appropriate substituted amine under catalytic conditions (e.g., ethanolic HCl; CuCl; Ln(III) ions) to provide the substituted amidine (IV).

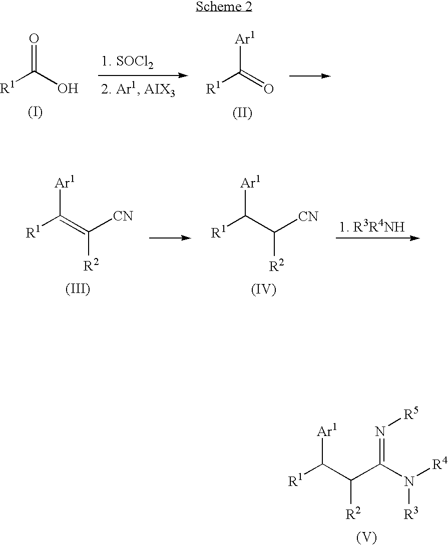

Acid (I) is converted into the acid chloride and treated with aluminum halide in the presence of an arene to give ketone (II). Treatment of ketone (II) with a dialkyl cyanomethylphosphonate under basic conditions provides the acrylonitrile derivative (III), which is reduced to propionitrile (IV). Treatment of propionitrile (IV) with the reagent formed by reaction of a trialkylaluminum with an amine gives, after hydrolysis, amidine (V).

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2nd. Ed., Wiley-VCH Publishers (1999); T. W. Greene and P. G. M. Wuts, Protective Groups in Organic Synthesis, 3rd. Ed., John Wiley and Sons (1999); L. Fieser and M. Fieser, Fieser and Fieser's Reagents for Organic Synthesis, John Wiley and Sons (1999); and L. Paquette, ed., Encyclopedia of Reagents for Organic Synthesis, John Wiley and Sons (1995), and subsequent editions thereof.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also be represented in multiple tautomeric forms, in such instances, the invention expressly includes all tautomeric forms of the compounds described herein (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

As used herein, the compounds of this invention, including the compounds of formulae described herein, are defined to include pharmaceutically acceptable derivatives or prodrugs thereof. A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention. Particularly favored derivatives and prodrugs are those that increase the bioavailability of the compounds of this invention when such compounds are administered to a mammal (e.g., by allowing an orally administered compound to be more readily absorbed into the blood) or which enhance delivery of the parent compound to a biological compartment (e.g., the brain or lymphatic system) relative to the parent species. Preferred prodrugs include derivatives where a group which enhances aqueous solubility or active transport through the gut membrane is appended to the structure of formulae described herein. See, e.g., Alexander, J. et al. Journal of Medicinal Chemistry 1988, 31, 318-322; Bundgaard, H. Design of Prodrugs; Elsevier: Amsterdam, 1985; pp 1-92; Bundgaard, H.; Nielsen, N. M. Journal of Medicinal Chemistry 1987, 30, 451-454; Bundgaard, H. A Textbook of Drug Design and Development; Harwood Academic Publ.: Switzerland, 1991; pp 113-191; Digenis, G. A. et al. Handbook of Experimental Pharmacology 1975, 28, 86-112; Friis, G. J.; Bundgaard, H. A Textbook of Drug Design and Development; 2 ed.; Overseas Publ.: Amsterdam, 1996; pp 351-385; Pitman, I. H. Medicinal Research Reviews 1981, 1, 189-214; Sinkula, A. A.; Yalkowsky. Journal of Pharmaceutical Sciences 1975, 64, 181-210; Verbiscar, A. J.; Abood, L. G Journal of Medicinal Chemistry 1970, 13, 1176-1179; Stella, V. J.; Himmelstein, K. J. Journal of Medicinal Chemistry 1980, 23, 1275-1282; Bodor, N.; Kaminski, J. J. Annual Reports in Medicinal Chemistry 1987, 22, 303-313.

The compounds of this invention may be modified by appending appropriate functionalities to enhance selective biological properties. Such modifications are known in the art and include those which increase biological penetration into a given biological compartment (e.g., blood, lymphatic system, nervous system), increase oral availability, increase solubility to allow administration by injection, alter metabolism and alter rate of excretion.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization.

The compounds of the formulae described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/g of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

The compositions delineated herein include the compounds of the formulae delineated herein, as well as additional therapeutic agents if present, in amounts effective for achieving a modulation of disease or disease symptoms, including ion channel-mediated disorders or symptoms thereof. References which include examples of additional therapeutic agents are: 1) Burger's Medicinal Chemistry & Drug Discovery 6^{th} edition, by Alfred Burger, Donald J. Abraham, ed., Volumes 1 to 6, Wiley Interscience Publication, NY, 2003; 2) Ion Channels and Disease by Francis M. Ashcroft, Academic Press, NY, 2000; and 3) Calcium Antagonists in Clinical Medicine 3^{rd} edition, Murray Epstein, MD, FACP, ed., Hanley & Belfus, Inc., Philadelphia, Pa., 2002. Additional therapeutic agents include but are not limited to agents for the treatment of cardiovascular disease (e.g., hypertension, angina, atrial fibrillation, prevention of stroke, heart failure, acute myocardial ischemia, etc), metabolic disease (e.g., syndrome X, diabetes, obesity), renal or genito-urinary disease (e.g, glomerular nephritis, urinary incontinence, nephrotic syndrome), and their disease symptoms. Examples of additional therapeutic agents for treatment of cardiovascular disease and disease symptoms include but are not limited to antihypertensive agents, ACE inhibitors, angiotensin II receptor antagonists, statins, β-blockers, antioxidants, anti-inflammatory drugs, anti-thrombotics, anti-coagulants or antiarrhythmics. Examples of additional therapeutic agents for treatment of metabolic disease and disease symptoms include but are not limited to ACE inhibitors, angiotensin II antagonists, fibrates, thiazolidinediones or sulphonylurea anti-diabetic drugs. Examples of additional therapeutic agents for treatment of renal and/or genitor-urinary syndromes and their symptoms include but are not limited to alpha-1 adrenergic antagonists (e.g., doxazosin), anti-muscarinics (e.g., tolterodine), norepinephrine/serotonin reuptake inhibitors (e.g., duloxetine), tricyclic antidepressants (e.g., doxepin, desipramine) or steroids.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

Also within the invention is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The invention will be further described in the following examples. It should be understood that these examples are for illustrative purposes only and are not to be construed as limiting this invention in any manner.

### EXAMPLE 1

### Oocyte Assay

Representative compounds of the formulae herein are screened for activity against calcium channel targets in an assay essentially as described in Neuron January 1997, 18(11): 153-166, Lin et. al.; J. Neurosci. Jul. 1, 2000, 20(13):4768-75, J. Pan and D. Lipsombe; and J. Neurosci., Aug. 15, 2001, 21(16):5944-5951, W. Xu and D. Lipscombe, using Xenopus oocyte heterologeous expression system. The assay is performed on various calcium channels (e.g., Caᵥ1.2 or Caᵥ1.3 subfamily) whereby the modulation of the calcium channel is measured for each compound.

### EXAMPLE 2

### HEK Assay

HEK-293T/17 cells are transiently transfected in a similar manner as described in FuGENE 6 Package Insert Version 7, April 2002, Roche Applied Science, Indianapolis, Ind. The cells are plated at 2.5×10⁵ cells in 2 mL in a 6-well plate in incubator for one night and achieve a 30-40% confluence. In a small sterile tube, add sufficient serum-free medium as diluent for FuGENE Transfection Reagent (Roche Applied Science, Indianapolis, Ind.), to a total volume of 100 μL. Add 3 μL of FuGENE 6 Reagent directly into this medium. The mixture is tapped gently to mix. 2 μg of DNA solution (0.8-2.0 μg/μL) is added to the prediluted FuGENE 6 Reagent from above. The DNA/Fugene 6 mixture is gently pipeted to mix the contents and incubated for about 15 minutes at room temperature. The complex mixture is then added to the HEK-293T/17 cells, distributing it around the well, and swirled to ensure even dispersal. The cells are returned to the incubator for 24 hrs. The transfected cells are then replated at density 2.5×10⁵ in a 35 mm dish with 5 glass coverslips and grow in low serum (1%) media for 24 hrs. Coverslips with isolated cells are then transferred into chamber and calcium channel (e.g., L-type, N-type, etc.) current or other currents for counter screening are recorded from the transiently transfected HEK-293T/17 cells.

The whole-cell voltage clamp configuration of the patch clamp technique is employed to evaluate voltage-dependent calcium currents essentially as described by Thompson and Wong (1991) J. Physiol., 439: 671-689. To record calcium channel (e.g., L-type, N-type, etc.) currents for evaluation of inhibitory potency of compounds (steady-state concentration-response analysis), five pulses of 20-30 ms voltage steps to about +10 mV (the peak of the current voltage relationship) are delivered at five Hz every 30 second from a holding potential at −100 mV. Compound evaluations were carried out essentially as described by Sah D W and Bean B P (1994) Mol Pharmacol. 45(1):84-92.

### EXAMPLE 3

### Formalin Test

Representative compounds of the formulae herein are screened for activity in the formalin test. The formalin test is widely used as a model of acute and tonic inflammatory pain (Dubuisson & Dennis, 1977 Pain 4:161-174; Wheeler-Aceto et al, 1990, Pain 40:229-238; Coderre et al, 1993, Pain 52:259-285). The test involves the administration to the rat hind paw of a dilute formalin solution followed by monitoring behavioral signs (i.e., flinching, biting and licking) during the “late phase” (11 to 60 minutes post injection) of the formalin response which reflects both peripheral nerve activity and central sensitization. Male, Sprague-Dawley rats (Harlan, Indianapolis, Ind.) weighing approximately 225-300 g are used with an n=6-8 for each treatment group.

Depending on pharmacokinetic profile and route of administration, vehicle or a dose of test compound is administered to each rat by the intraperitoneal or oral route 30-120 minutes prior to formalin. Each animal is acclimated to an experimental chamber for 60 minutes prior to formalin administration, which is 50 μL of a 5% solution injected subcutaneously into the plantar surface of one hind paw using a 300 μL microsyringe and a 29 gauge needle. A mirror is angled behind the chambers to enhance the views of the animals' paws. The number of flinches (paw lifts with or without rapid paw shaking) and the time spent biting and/or licking the injured hind paw are recorded for each rat for 2 continuous minutes every 5 minutes for a total of 60 minutes after formalin administration. A terminal blood sample is harvested for analysis of plasma compound concentrations. Between groups comparisons of the total number of flinches or time spent biting and/or licking during the early or late phase are conducted using one-way analysis of variance (ANOVA).

### EXAMPLE 4

Representative compounds of the formulae herein are evaluated for activity against calcium channel targets.

### Compound 1

### 2,3-Bis-(4-tert-butyl-phenyl)-propionamidine

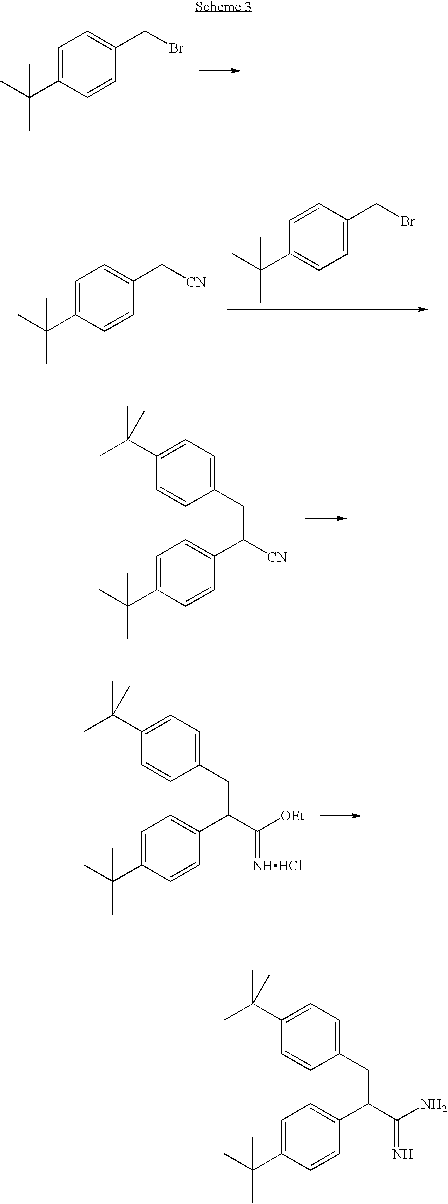

### Part 1

### Preparation of (4-tert-Butyl-phenyl)-acetonitrile

To a solution of potassium cyanide (5.3 g, 81.6 mmol) in 1:6 water/ethanol (420 mL) was added 4-(tert-butyl)benzyl bromide (18.5 g, 81.6 mmol) and the mixture stirred at reflux for 17 h. After cooling to room temperature the resulting white precipitate was removed by filtration. The filtrate was concentrated in vacuo, the residue taken up in ethyl acetate/water and extracted with ethyl acetate. The organics were dried and concentrated in vacuo to give a colorless oil. Purification by chromatography (SiO₂, 5% ethyl acetate in n-hexane) gave (4-tert-Butyl-phenyl)-acetonitrile (14.0 g, 80.8 mmol) as a colorless oil.

### Part 2

### Preparation of 2,3-Bis-(4-tert-butyl-phenyl)-propionitrile

To a solution of (4-tert-Butyl-phenyl)-acetonitrile (48.3 g, 279 mmol) in tetrahydrofuran (600 mL) at −78° C. was added lithium bis(trimethylsilyl)amide (335 ml, 335 mmol, 1 M solution in tetrahydrofuran) with stirring. After 1 h 4-(tert-butyl)benzyl bromide (63.4 g, 279 mmol) was added dropwise and the mixture stirred for 16 h while warming to room temperature. The mixture was quenched with water, concentrated in vacuo, the residue taken up in ethyl acetate/water and extracted with ethyl acetate. The organics were dried and concentrated in vacuo to give an off-white solid. Crystallization from ethyl acetate/hexanes gave 2,3-Bis-(4-tert-butyl-phenyl)-propionitrile (57.5 g, 180 mmol) as a white crystalline solid.

### Part 3

### Preparation of 2,3-Bis-(4-tert-butyl-phenyl)-propionimidic acid ethyl ester; hydrochloride

Into a solution of 2,3-Bis-(4-tert-butyl-phenyl)-propionitrile (0.50 g, 1.56 mmol) in 1:1 ethanol/diethyl ether (20 mL) at 0° C. was bubbled HCl gas over 15 min. The reaction was stoppered and warmed to room temperature for 6 h. Concentration in vacuo gave crude 2,3-Bis-(4-tert-butyl-phenyl)-propionimidic acid ethyl ester; hydrochloride which was used without further purification.

### Part 4

### Preparation of 2,3-Bis-(4-tert-butyl-phenyl)-propionamidine

2,3-Bis-(4-tert-butyl-phenyl)-propionimidic acid ethyl ester; hydrochloride (0.10 g, 0.27 mmol) was treated with 2M ammonia in 2-propanol (10 mL), sealed and was heated at 40C overnight. The reaction vessel was cooled, opened, and the solution concentrated under vacuum to give a white residue. The residue was triturated with a diethyl ether/methanol (10:1/v:v) solution, filtered, and dried under high vacuum to give 2,3-Bis-(4-tert-butyl-phenyl)-propionamidine hydrochloride (0.04 g, 0.1 immunol) as a white solid.

### Compound 2

### 3,3-Bis-(4-tert-butyl-phenyl)-propionamidine

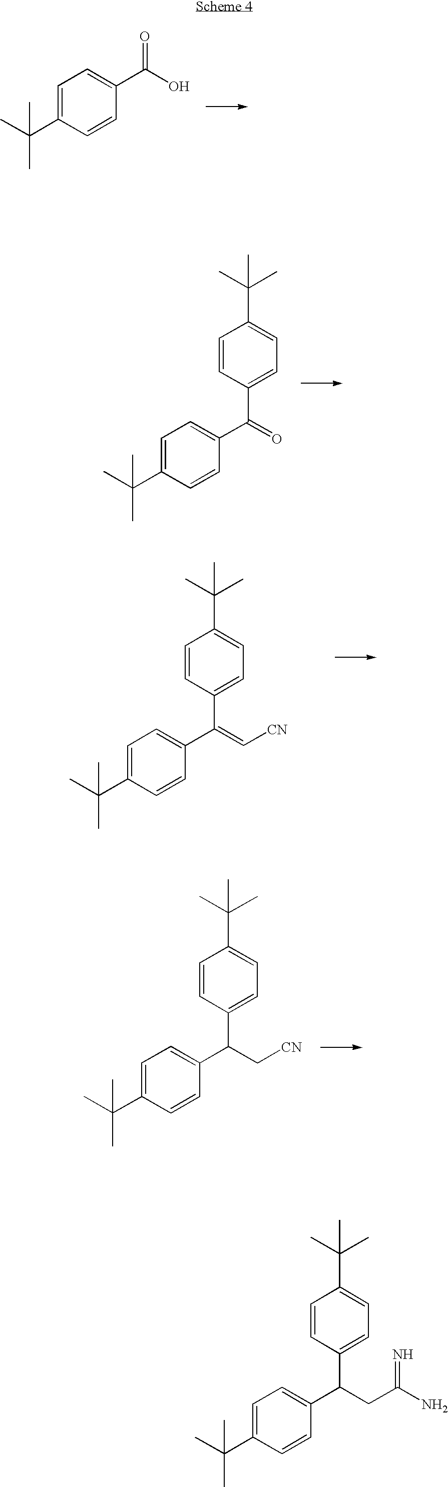

### Part 1

### Preparation of (Bis-(4-tert-butyl-phenyl)-methanone

To 4-tert-butylbenzoic acid (5.2 g, 29.2 mmol) was added thionyl chloride (6.3 g, 53.0 mmol) and the mixture stirred at 80° C. for 15 h. After cooling to room temperature excess thionyl chloride was removed in vacuo to give the acid chloride as a light yellow oil. To the crude acid chloride was added tert-butylbenzene (9.4 g, 70.2 mmol) followed by aluminum chloride (7.8 g, 58.5 mmol) and the mixture stirred at 80° C. for 2 h. The mixture was cooled to room temperature, poured onto ice, treated with conc. HCl (35 ml), and extracted with ethyl acetate. The organics were dried and concentrated in vacuo to give a light brown solid. Re-crystallization from ethanol gave (Bis-(4-tert-butyl-phenyl)-methanone (5.4 g, 18.3 mmol) as an off-white crystalline solid.

### Part 2

### Preparation of 3,3-Bis-(4-tert-butyl-phenyl)-acrylonitrile

To a solution of (bis-(4-tert-butyl-phenyl)-methanone (1.2 g, 7.0 mmol) and diethyl cyanomethylphosphonate (1.5 g, 8.4 mmol) in tetrahydrofuran (30 mL) at room temperature was added sodium hydride (0.4 g, 10.5 mmol, 60% dispersion in oil) and the mixture stirred for 16 h. The mixture was quenched with 0.1 N HCl and extracted with diethyl ether. The organics were dried and concentrated in vacuo to give a red oil. Purification by column chromatography (SiO₂, 5% ethyl acetate in n-hexane) gave 3,3-Bis-(4-tert-butyl-phenyl)-acrylonitrile (1.0 g, 3.2 mmol) as a white crystalline solid.

### Part 3

### Preparation of 3,3-bis-(4-tert-butyl-phenyl)-propionitrile

A mixture of 3,3-bis-(4-tert-butyl-phenyl)-acrylonitrile (4 g, 12.6 mmol) and 10% Pd/C (1.2 g) in ethyl acetate (10 mL) and ethanol (10 mL) was hydrogenated at room temperature at an initial pressure of 42 psi. After 2 days, the mixture was passed through a pad of Celite. The filtrate was applied to column chromatography (SiO₂, 5% ethyl acetate in n-hexane) to give 3,3-Bis-(4-tert-butyl-phenyl)-propionitrile (3.5 g, 11.0 mmol) as a white solid.

### Part 4

### Preparation of 3,3-bis-(4-tert-butyl-phenyl)-propionamidine

To a suspension of ammonium chloride (0.3 g, 5.6 mmol) in toluene (10 mL) at 0° C. was added trimethylaluminum (2.7 ml, 5.32 mmol, 2.0 M in hexanes) dropwise with stirring. After complete addition the cooling bath was removed and the mixture stirred an additional 1.5 h. A solution of 3,3-bis-(4-tert-butyl-phenyl)-propionitrile (1.0 g, 3.1 mmol) in toluene (10 mL) and dichloromethane (1 mL) was added dropwise and the mixture was heated at 80° C. for 16 h. The slurry was filtered through a pad of Celite and washed with 20% methanol in dichloromethane. The filtrate was concentrated under vacuum and applied to column chromatography (SiO₂, 20% methanol in dichloromethane). The fractions containing the product were combined, evaporated to dryness, and washed with ethyl acetate. 3,3-Bis-(4-tert-butyl-phenyl)-propionamidine (200 mg, 0.54 mmol) was obtained as the hydrochloride salt.

Compounds in the tables herein are prepared in a manner similar as described above and in the general schemes.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound of formula (IA) or pharmaceutical salt thereof 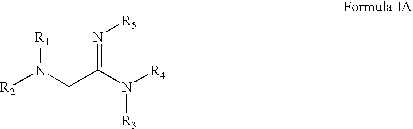
wherein,
R¹ is (CH₂)ₘAr¹;
Ar¹ is aryl, heteroaryl, heterocyclyl or cycloalkyl, each optionally substituted with one or more R⁹;
m is 0, 1, 2, 3, 4 or 5;
R² is (CH₂)ₙAr²
n is 0, 1, 2, or 3;
Ar² is aryl or heteroaryl, each optionally substituted with one or more R⁹;
R³ is H, alkyl, or (CH₂)ₚ;
p is 0, 1, 2 or 3;
Z is OCH₂CH₂OH, NR⁶R⁷, OR⁴, or Ar³;
Ar³ is cycloalkyl, heterocyclyl, aryl, or heteroaryl, each optionally substituted with one or more R⁹;
or R³ and R⁴ taken together with the nitrogen atom to which they are attached form a 3 to 6 membered-ring, having carbon atoms and optionally in addition to the aforementioned nitrogen atom 1 or 2 additional heteroatoms that are NR¹⁰, O or S, wherein the ring formed by R³ and R⁴ can be substituted by 1-3 R⁹;
each R⁴ is independently H or lower alkyl;
R⁵ is H or lower alkyl;
each R⁶ is independently hydrogen or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁷ is independently hydrogen, (CH₂)_{q}Ar⁴, or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁸ is independently (CH₂)_{q}Ar⁴ or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each Ar⁴ is independently aryl or heteroaryl, each optionally substituted with one to three substituents independently selected from halogen, OH, C₁-C₄ alkoxy, NH₂, C₁-C₄ alkylamino, C₁-C₄ dialkylamino or C₃-C₆ cycloalkyl;
each q is independently 0 or 1; and
each R⁹ is independently halogen, CN, NO₂, OR⁶, SR⁶, S(O)₂OR⁶, NR⁶R⁷, alkyl, hydroxyalkyl, cycloalkyl, Ar⁴, Ar⁴alkyl, C₁-C₂ perfluoroalkyl, C₁-C₂ perfluoroalkoxy, 1,2-methylenedioxy, C(O)OR⁶, C(O)NR⁶R⁷, OC(O)NR⁶R⁷, NR C(O)NR⁶R⁷, C(NR⁶)NR⁶R⁷, NR⁶C(NR⁷)NR⁶R⁷, S(O)₂NR⁶R⁷, R⁸, C(O)R⁸, NR⁶C(O)R⁸, S(O)R⁸, or S(O)₂R⁸; and
each R¹⁰ is independently alkyl, aryl or aralkyl, each optionally substituted with one or more R⁹.

2. The compound of claim 1, wherein:
R¹ is aryl or (CH₂)aryl, each optionally substituted with one or more R⁹;
R² is aryl or heteroaryl, each optionally substituted with one or more R⁹;
R³ is (CH₂)ₚZ;
R⁴ is H; and
R⁵ is H;
wherein R⁹, Z, and p are as defined in claim 1.

3. (canceled)

4. The compound of claim 1, wherein Z is Ar³.

5-6. (canceled)

7. The compound of claim 1, wherein R³ and R⁴ taken together with the nitrogen atom to which they are attached form a 3 to 6 membered-ring, having carbon atoms and optionally in addition to the aforementioned nitrogen atom 1 or 2 additional heteroatoms that are NR¹⁰, O or S, wherein the ring formed by R³ and R⁴ can be substituted by 1-3 R⁹.

8. The compound of claim 1, wherein when R³, R⁴ and R⁵ are simultaneously H, R¹ and R² are not simultaneously unsubstituted phenyl and unsubstituted benzyl.

9. The compound of claim 1, wherein when R³, R⁴ and R⁵ are simultaneously H, R¹ is (CH₂)ₘAr¹; and Ar¹ is aryl, heteroaryl, heterocyclyl or cycloalkyl, each substituted with one or more R⁹.

10. A compound of formula (IB) or pharmaceutical salt thereof 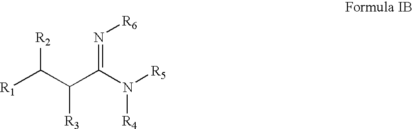
wherein,
R¹ is (CH₂)ₘAr¹;
Ar¹ is aryl, heteroaryl, heterocyclyl or cycloalkyl, each optionally substituted with one or more R¹⁰;
m is 0, 1, 2, 3, 4 or 5;
R³ is (CH₂)ₚAr²;
p is 0, 1 or 2;
Ar² is aryl, or heteroaryl, each optionally substituted with one or more R¹⁰;
R² is H;
R⁴ is H, alkyl, (CH₂)ₘZ, or C(O)R⁵;
Z is OCH₂CH₂OH, NR⁷R⁸, OR⁵, or Ar³;
Ar³ is cycloalkyl, heterocyclyl, aryl, or heteroaryl, each optionally substituted with one or more R¹⁰;
or R⁴ and R⁵ taken together with the nitrogen atom to which they are attached form a 3 to 6 membered-ring, having carbon atoms and optionally in addition to the aforementioned nitrogen atom 1 or 2 additional heteroatoms that are NR¹¹, O or S, wherein the ring formed by R⁴ and R⁵ can be substituted by 1-3 R¹⁰;
each R⁵ is independently H or lower alkyl;
R⁶ is H or lower alkyl;
or R⁵ and R⁶ taken together are —(CR¹²R¹³)ₙ—, where n is 2 or 3;
each R⁷ is independently hydrogen or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁸ is independently hydrogen, (CH₂)_{q}Ar⁴, or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each R⁹ is independently (CH₂)_{q}Ar⁴ or lower alkyl optionally substituted with one or more substituent independently selected from halogen, OH, C₁-C₄ alkoxy, NH₂, C₁-C₄ alkylamino, C₁-C₄ dialkylamino or C₃-C₆ cycloalkyl;
each Ar⁴ is independently aryl or heteroaryl, each optionally substituted with one to three substituents independently selected from halogen, OH, C₁-C₄ alkoxy, NH₂, C₁-C₄ alkylamino, C₁-C₄ dialkylamino or C₃-C₆ cycloalkyl;
each q is 0 or 1;
each R¹⁰ is independently halogen, CN, NO₂, OR⁷, SR⁷, S(O)₂OR⁷, NR⁷R⁸, alkyl, hydroxyalkyl, cycloalkyl, Ar⁴, Ar⁴alkyl, C₁-C₂ perfluoroalkyl, C₁-C₂ perfluoroalkoxy, oxo, 1,2-methylenedioxy, C(O)OR⁷, C(O)NR⁷R⁸, OC(O)NR⁷R⁸, NR⁷C(O)NR⁷R⁸, C(NR⁷)NR⁷R⁸, NR⁷C(NR⁸)NR⁷R⁸, S(O)₂NR⁷R⁸, R⁹, C(O)R⁹, NR⁷C(O)R⁹, S(O)R⁹, or S(O)₂R⁹;
each R¹¹ is independently alkyl, aryl or aralkyl, each optionally substituted with one or more R¹⁰;
each R¹² is independently H, alkyl, or aryl; and
each R¹³ is independently H, alkyl, or aryl.

11. The compound of claim 10, wherein:
R¹ is (CH₂)aryl, optionally substituted by one or more R¹⁰;
R³ is aryl, optionally substituted by one or more R¹⁰;
R⁴ is (CH₂)ₘZ;
R⁵ is H; and
R⁶ is H,
wherein R¹⁰, Z, and m are as defined in claim 10.

12. The compound of claim 11, wherein Z is Ar³.

13. The compound of claim 12, wherein Ar³ is aryl, heteroaryl, or heterocyclyl, each optionally substituted with one or more R¹⁰.

14-15. (canceled)

16. The compound of claim 10, wherein:
R⁵ and R⁶ taken together are —(CR¹²R¹³)ₙ—, where n is 2 or 3;
R¹ is Ar¹ or (CH₂)Ar¹; and
R³ is aryl or heteroaryl, each optionally substituted with one or more R¹⁰,
where R¹², R¹³ and Ar¹ are as defined in claim 10.

17. The compound of claim 1, wherein the compound is one of those delineated in Table A-1A or A-1B.

18. The compound of claim 10, wherein the compound is one of those delineated in Table B-1A or B-1B.

19. A method for treating a disease or disease symptom in a subject comprising administering an effective amount of a compound of claim 1 or pharmaceutical salt thereof.

20. The method of claim 19, wherein the disease or disease symptom is angina, hypertension, congestive heart failure, myocardial ischemia, atrial fibrillation, diabetes mellitus, urinary incontinence, overactive bladder, pulmonary disease, cognitive function, or a nervous system disorder.

21. The method of claim 19, wherein the disease or disease symptom is modulated by calcium channel Caᵥ1.

22. The method of claim 19, wherein the disease or disease symptom is modulated by calcium channel Caᵥ1.2 or Caᵥ1.3.

23-26. (canceled)

27. A method for treating a disease or disease symptom in a subject comprising administering an effective amount of a compound of claim 10 or pharmaceutical salt thereof.

28. The method of claim 27, wherein the disease or disease symptom is angina, hypertension, congestive heart failure, myocardial ischemia, atrial fibrillation, diabetes mellitus, urinary incontinence, overactive bladder, pulmonary disease, cognitive function, or a nervous system disorder.

29. The method of claim 27, wherein the disease or disease symptom is modulated by calcium channel Caᵥ1.
